(19) United States
(12) Patent Application Publication
(10) Pub. No.: US 2010/0282245 A1
(43) Pub. Date: Nov. 11, 2010

(54) Detection of nitric oxide

(76) Inventors: Alexander Star, Pittsburgh, PA (US); Oleksander Kuzmych, Pittsburgh, CA (US)

Correspondence Address: HENRY E. BARTONY, JR, BARTONY & ASSOCIATES LLC, P.O. BOX 910, BUTLER, PA, 16003-0910, US

(21) Appl. No.: 12/008,800

(22) Filed: Jan. 14, 2008

Related U.S. Application Data
(60) Provisional application No. 60/880,192, filed on Jan. 12, 2007.

(51) Int. Cl. A61M 11/00; G01N 33/497; G01N 33/00

(52) U.S. Cl. 128/200.14; 422/83; 422/84; 422/98; 436/116; 977/953

(57) ABSTRACT

A system for the detection of nitric oxide in a gas sample includes a converter for oxidation of nitric oxide to nitrogen dioxide, a nitrogen dioxide sensor including nanostructures and a filtering device to remove at least carbon dioxide from the gas sample positioned upstream of the converter. The nitrogen dioxide sensor can, for example, include a recognition layer on the nanostructures adapted to enhance sensitivity to nitrogen dioxide. A method for detecting nitric oxide in exhaled breath includes detecting nitric oxide in the exhaled breath using a nitric oxide sensor including nanostructures. Another method for detecting nitric oxide in exhaled breath includes filtering the breath to remove at least carbon dioxide from the breath, oxidizing nitric oxide in the exhaled breath to nitrogen dioxide and detecting the nitrogen dioxide using a nitrogen dioxide sensor including nanostructures.

### CROSS-REFERENCE TO RELATED APPLICATION

This application claims benefit of U.S. Provisional Patent Application Ser. No. 60/880,192, filed Jan. 12, 20007, the disclosure of which is incorporated herein by reference.

### BACKGROUND OF THE INVENTION

The present invention relates to the detection of nitric oxide (NO) and, particularly, to the detection of nitric oxide in exhaled gases or breath.

The following information is provided to assist the reader to understand the invention disclosed below and the environment in which it will typically be used. The terms used herein are not intended to be limited to any particular narrow interpretation unless clearly stated otherwise in this document. References set forth herein may facilitate understanding of the present invention or the background of the present invention. The disclosure of all references cited herein are incorporated by reference.

Nitric oxide (NO) plays an extremely important role in lung function. It is, for example, responsible for pulmonary vascular tone and neurotransmission. NO also mediates response to inflammation. NO is formed in the lungs of mammals and can be detected in exhaled air. The exhaled breath of healthy individuals exhibits a concentration of NO in the range or approximately 10-20 ppbv (parts per billion by volume). However, people with asthma can, for example, exhibit an increased level of NO in exhaled breath (for example, in the range of approximately 70-100 ppbv). See Gustafsson et al., Endogenous nitric oxide is present in the exhaled air of rabbits, guinea pigs and humans, Biochem. Biophys. Res. Commun. 1991, 181, 852-857. Exhaled air from human breath also contains approximately 5.6% CO₂, approximately 16% O₂, H₂O(RH of approximately 100%) and traces of organic substances, which can overshadow nitric oxide.

Various analytical methods for detection of NO include: amperometric methods, gas phase chemiluminescence methods, fluorescence methods, Electron Spin Resonance (ESR) methods, and Infrared Spectroscopy (IR) methods. Chemiluminescense methods have, for example, been used to detect the relatively low concentrations of NO in exhaled breath. In one such technique, NO is first oxidized to nitrogen dioxide (NO₂), and NO₂ is detected by it chemiluminescent reaction with an alkaline liminol/H₂O₂ solution. Robinson, J. K. et al., Luminol/H₂O₂ Chemiluminescence Detector for the Analysis of Nitric Oxide in Exhaled Breath, Anal. Chem. 1999, 71, 5131-5136. The two reagents are pumped into a hollow fiber gas-liquid exchange module. A stream of exhaled breath is directed into the gas-liquid exchange module after passing through a converter to oxidize NO in the exhaled breath to NO₂. Light emitted as a result of the reaction of NO₂ with luminol/H₂O₂ is detected by a light sensor such as a photomultiplier tube. Current analytical methods for detecting NO suffer from a number of drawbacks including, for example, large size, high expense and operational complexity.

Recently, a number of gas sensors have been developed for the detection of gases (including, for example, ammonia (NH₃), NO₂, carbon monoxide (CO), carbon dioxide (CO₂), hydrogen sulfide (H₂S), hydrogen (H₂), methane (CH₄) and alcohol vapors) based upon on electronic detection using nanostructure-based sensors such as those including nanotube field effect transistor (NTFET) devices. See, for example, Qui, P. et al., Toward large Arrays of multiplex functionalized carbon nanotube sensors for highly sensitive and selective molecular detection, Nano Lett. 2003, 3, 347-351; Kong J., et al., Nanotube molecular wires as chemical sensors, Science 2000, 287, 622; Star A., et al., Gas sensor array based on metal-decorated carbon nanotubes, J. Phys. Chem. B. 2006, 110, 21014; Star A., et al. Nanoelectronic carbon dioxide sensor, Adv. Mater. 2004, 16, 2049; Kong J., et al. Functionalized carbon nanotubes for molecular hydrogen sensor, Adv. Mater., 2001, 13, 1384; Lu Y., et. al. Room temperature methane detection using palladium loaded single-walled carbon nanotube sensors, Chem. Phys. Lett. 2004, 391, 344; Someya T., et. al., Alcohol Vapor Sensors Based on Single-Walled Carbon Nanotube Field Effect Transistors, Nano Lett 2003, 3, 877; U.S. Patent Application Publication Nos. 2005/0279987 and 2005/129,573.

It is desirable to develop improved devices, systems and methods for the detection of NO and, particularly, for the detection of NO in breath for medical applications such as asthma diagnostics.

### SUMMARY OF THE INVENTION

In one aspect, the present invention provides a system for the detection of nitric oxide in a gas sample (for example, exhaled breath) including a converter for oxidation of nitric oxide to nitrogen dioxide, a nitrogen dioxide sensor including at least one nanostructure and a filtering device to remove at least carbon dioxide from the gas sample positioned upstream of the converter. In several embodiments, the nitrogen dioxide sensor includes a network of carbon nanotubes. For example, the sensor can include a source electrode is in contact with the network of carbon nanotubes and a drain electrode in contact with the network of carbon nanotubes. In several embodiments, the nitrogen dioxide sensor includes at least one nanotube field effect transistor (NTFET) device.

The nitrogen dioxide sensor can, for example, include a recognition layer in contact with the nanostructure(s) to enhance sensitivity to nitrogen dioxide. The recognition layer can, for example, include at least one polymer, a metal or a metal compound. A recognition layer including a polymer having amino functionality can, for example, enhance sensitivity to nitrogen dioxide. Examples of suitable polymers include, but are not limited to, polyethyleneimine (PEI), polyamidoamine (PAMAM), Polydi(carbazol-3-yl)phenylamine, nylon or poly(N-isopropylacylamide) (PNIMAM). In several embodiments, the recognition layer includes polyethyleneimine.

The filtering device can, for example, be operable to remove interferents other than carbon dioxide from the gas sample. The filtering device can, for example, remove acid gasses.

The converter can, for example, include a catalyst to effect oxidation of nitric oxide such as chromium trioxide.

The system can include at least one other sensor including nanostructures (for example, a network of carbon nanotubes) to sense a gas other than nitrogen dioxide. The system can include a plurality of other sensors including nanostructures to sense gases other than nitrogen dioxide. Example of other sensors that can be included in the system are carbon monoxide sensors and carbon dioxide sensors.

Other types of sensors can also be included in the system. For example the system can further include a flow meter to measure rate of flow. A sensor to measure volume of exhaled gas can be included. A flow meter can, for example, be a peak flow meter. Peak flow meter can measure rate of flow and volume of exhaled gas.

At least the nitrogen dioxide sensor can be disposable. Moreover, the entire system or any portion thereof can be disposable on, for example, a per-use or a per-patient basis.

The nitrogen dioxide sensor can, for example, be in communicative connection with a controller which is operable to or adapted to determine level of nitric oxide based at least in part on output of the nitrogen dioxide sensor. The controller can, for example, be in operative connection with a memory that, for example, comprises a lookup table or an algorithm that relates an output signal from the sensor to a level of nitric oxide. The controller can be in communicative connection with a display to display information related to a determined level of nitric oxide.

The system can further include a mouthpiece system in fluid connection with the converter and the nitrogen dioxide sensor. The mouthpiece can, for example, be in connection with (for example, removably connectible) to a medication dosing system. In one embodiment, the controller provides information (for example, to the medication dosing system) related to the determined level of nitric oxide. A dose of medication can be determined from the determined level of nitric oxide.

The converter and the nitrogen dioxide sensor can, for example, be components of the mouthpiece system. The controller can also be a component of the mouthpiece system.

The filtering device, the converter, and the nitrogen dioxide can, for example, be components of a medication dosing system such as an inhaler.

The system can further include a system to reduce relative humidity of the gas sample prior to contact with the sensor.

In another aspect, the present invention provides system for the detection of nitric oxide in a gas sample including a converter for oxidation of nitric oxide to nitrogen dioxide and a nitrogen dioxide sensor comprising at least one nanostructure.

In another aspect, the present invention provides a system for the detection of nitric oxide in a gas sample (such as exhaled breath) including a nitric oxide sensor including at least one nanostructure.

In another aspect, the present invention provides a method for detecting nitric oxide in a gas sample including: filtering the gas sample to remove at least carbon dioxide from the gas sample; after filtering the gas sample, oxidizing nitric oxide in the gas sample to nitrogen dioxide and detecting the nitrogen dioxide using a nitrogen dioxide sensor including at least one nanostructure.

In a further aspect, the present invention provides a method for detecting nitric oxide in exhaled breath including detecting nitric oxide in the exhaled breath using a nitric oxide sensor including at least one nanostructure.

In still a further aspect, the present invention provides a method for detecting nitric oxide in exhaled breath including: filtering the exhaled breath to remove at least carbon dioxide from the gas sample, after filtering the exhaled breath, oxidizing nitric oxide in the exhaled breath to nitrogen dioxide and detecting the nitrogen dioxide using a nitrogen dioxide sensor including nanotubes.

The present invention, along with the attributes and attendant advantages thereof, will best be appreciated and understood in view of the following detailed description taken in conjunction with the accompanying drawings.

### BRIEF DESCRIPTION OF THE DRAWINGS

FIG. 1A illustrates a schematic representation of an embodiment of a NO detection system of the present invention in which NO is converted to NO₂, which is then sensed by a nanotube field effect transistor (NTFET) device.

FIG. 1B illustrates an embodiment of the NTFET device of the system of FIG. 1A.

FIG. 1C illustrates a graph of S-D conductance as a function of gate voltage (G-V_{G}) of an NTFET device of the present invention, including an inset scanning electron microscopy (SEM) image showing part of two interdigitated electrodes of 5 μm pitch connecting the SWNT network after coating with polymer.

FIG. 2 illustrates a schematic representation of a gas dilution and testing system (wherein gas lines were stainless steel) used in studies of the present invention.

FIG. 3A illustrates a graph of conductance versus time dependence of a bare carbon nanotube field effect transistor (NTFET) device of the present invention exposed to four short pulses of nitric oxide (5 PPM in N₂) in the absence of and in the presence of a catalyst to convert NO to NO₂.

FIG. 3B illustrates a comparison of the response of a NTFET device of the present invention to single pulse of nitrogen dioxide and of nitric oxide.

FIG. 4A illustrates the dynamic range of NO sensors of the present invention based on bare and polymer-coated NTFET devices in setting forth a graph of conductance versus time dependence of a bare NTFET device of the present invention and a PEI-coated NTFET device of the present invention exposed to six short NO gas pulses in a concentration range between 0.2 PPM and 5 PPM.

FIG. 4B illustrates a graph of conductance versus time dependence of a bare NTFET device of the present invention and a PEI-coated NTFET device of the present invention exposed to six short NO gas pulses in a concentration range between 0.01 PPM and 0.3 PPM.

FIG. 4C illustrates calibration curves of a bare NTFET device of the present invention and a PEI-coated NTFET device of the present invention.

FIG. 5A illustrates conductance as a function of time wherein an NTFET device of the present invention was subjected to 0.1 ppm to 5 ppm pulsed CrO₃-converted NO in N₂ at 30% RH.

FIG. 5B illustrated the response of the device of FIG. 5A (ΔG in μS) at 0%, 30%, and 78% RH.

FIG. 5C illustrates conductance as a function of time for a PEI-coated NTFET device of the present invention subjected to 0.08 ppm to 0.15 ppm pulsed CrO3-converted NO in N₂ at 0% and 30% RH.

FIG. 5D illustrates conduction as a function of time for another PEI-coated NTFET device subjected to 0.1 ppm to 0.15 ppm pulsed CrO₃-converted NO in N₂ at 30% RH.

FIG. 6A illustrates a graph of conductance versus time dependence of a PEI-coated NTFET device of the present invention exposed to five short pulses of NO gas over a concentration range between 2 PPB and 60 PPB in 100% nitrogen atmosphere.

FIG. 6B illustrates a graph of conductance versus time dependence of a PEI-coated NTFET device of the present invention exposed to five short pulses of NO gas over a concentration range between 2 PPB and 60 PPB in 6% carbon dioxide.

FIG. 6C illustrates a graph of conductance versus time dependence of a PEI-coated NTFET device of the present invention exposed to five short pulses of NO gas over a concentration range between 2 PPB and 60 PPB in 16% oxygen balanced with nitrogen.

FIG. 6D illustrates a graphical representation of signal versus concentration of NO for a bare NTFET device in a 100% N₂ environment (♦) and in 94% N₂ and 6% CO₂ (▪).

FIG. 6E illustrates a graphical representation of signal versus concentration of NO for a PEI coated NTFET device in 100% N₂ (♦), in 94% N₂ and 6% CO₂ with a 5.0 g ASCARITE scrubber (▴), and in 94% N₂ and 6% CO₂ with no scrubber (▪).

FIG. 7 illustrates SEM images showing multiple interdigitated device geometries present on the NTFET chips (center) and enlarged sections thereof (right and left) showing selective Au deposition on device a₁, selective Pd deposition on device b₁, and selective Pt deposition on device c₂, while the complementary degenerate devices remained bare, as shown with device b₂.

FIG. 8A illustrates G-V_{G} transfer characteristics of Au-decorated device a₁ before and after selective Au deposition thereon.

FIG. 8B illustrates G-V_{G} transfer characteristics of bare device a₂ before and after selective Au deposition on device a₁.

FIG. 8C illustrates G-V_{G} modification of Pt-decorated devices c₁ and c₂ wherein different deposition times were used (devices c₁ and c₂ were held at −1.0 V for 10 and 20 s, respectively).

FIG. 9A illustrates G-V_{G} response of bare device a₂ to 10 ppm NO gas in N₂.

FIG. 9B illustrates G-V_{G} response of Au-decorated device a₁ to 10 ppm NO gas in N₂, wherein increased gate voltage shift indicates increased electron transfer into the SWNT network of the Au-decorated device a₁.

FIG. 10 illustrates a relationship between metal work function and absolute value of observed gate voltage shift in metal-decorated NTFET, wherein each metal was used to decorate two devices with a 10 s deposition time (solid labels) and a 20 s deposition time (hollow labels).

FIG. 11A illustrates an embodiment of a mouthpiece or mask including a nanotube sensor of the present invention associated therewith or incorporated therein.

FIG. 11B illustrates an embodiment of a sensor system of the present invention in fluid connection with a disposable flow path.

FIG. 12 illustrates an embodiment of a system of the present invention including a mouthpiece which can be attachable to, for example, a metered-dose inhaler, wherein the mouthpiece includes a sensor system of the present invention.

### DETAILED DESCRIPTION OF THE INVENTION

In several representative embodiments, sensors of the present invention are described as including one or more representative nanostructures including one or more single-wall carbon nanotubes (SWNTs), (for example, a network of SWNTs). As clear to those skilled in the art, various other nanostructures are suitable for use in the present invention. Such nanostructures include, for example, multiple-wall nanotubes, nanowires, nanofibers, nanorods, nanospheres, or the like, or mixtures of such nanostructures. Moreover, in addition to carbon, those skilled in the art will appreciate that the nanostructures of the present invention can be formed of boron, boron nitride, and carbon boron nitride, silicon, germanium, gallium nitride, zinc oxide, indium phosphide, molybdenum disulphide, silver, and/or other suitable materials.

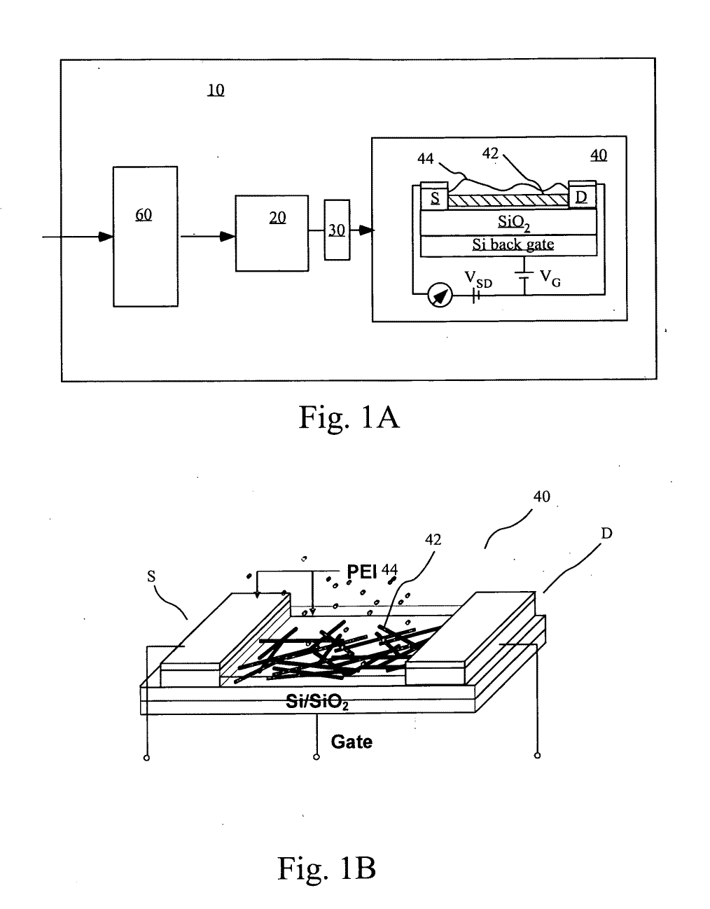

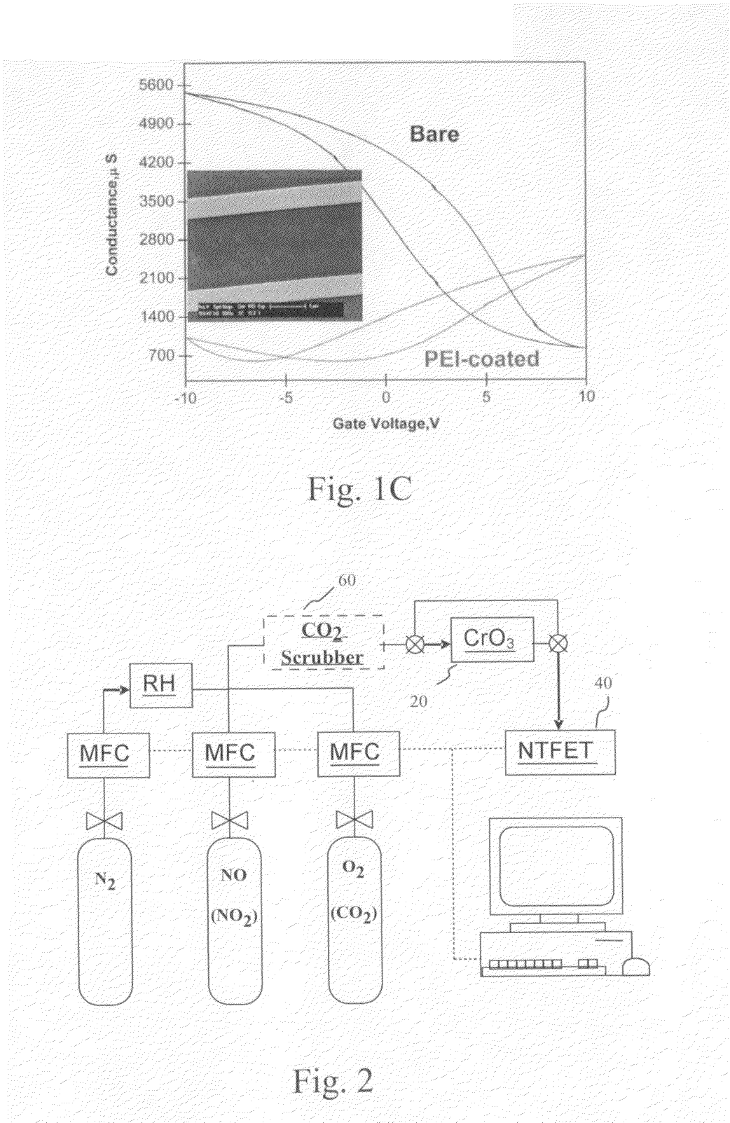

In single-walled carbon nanotubes, all carbon atoms are located on the surface where current flows, making a stable conduction channel that is extremely sensitive to a surrounding chemical environment. Nanotubes, including SWNT's, have the ability to change conductance in response to interaction with (for example, absorption of) different gases. This idea is, for example, implemented in a SWNT based field-effect transistor device, where a semiconducting SWNT or network of SWNTs is disposed upon a substrates and contacted by two metal (for example, Au/Ti) electrodes representing a source (S) and a drain (D) (see, for example, FIGS. 1A and 1B). The field effect transistor device can, for example, include a Si back gate separated by a SiO₂ and/or Si₃N₄ insulating layer in a FET-configured circuit. FIG. 1C shows S-D conductance versus gate voltage (G-V_{G}), the transfer characteristic of typical NTFET devices used in this study.

Measurements made with NTFETs composed of random networks of SWNTs can be advantageous because random network NTFETs are less prone to failure as a result of the large number of conduction pathways. Additionally, while random network NTFETs may not provide information on individual nanotube response, as with singly isolated SWNT FETs, they possess an intrinsic averaging effect in that they remove nanotube-to-nanotube variation as a result of the combined response of the entire network

As an analyte comes into contact with the device surface, SWNT conductance is modified to produce a detection signal. This modification can take the form of one of two types. The first type of modification takes the form of a charge transfer mechanism. The analyte can either donate an electron or withdrawal an electron. The second type of modulation takes the form of a scattering potential where the analyte disrupts the flow of electrons or holes on the nanotube surface, depending upon the characteristics of the device, either n-type (electron charge carriers) or p-type (hole carriers). The change in conductance is typically monitored versus time or applied gate voltage.

In several studies of the present invention, NTFET devices manufactured by Nanomix Inc., of Emeryville, California were used. NTFET devices were prepared using consecutive chemical vapor deposition (CVD) and photolithography process as, for example, described in Star, A., et al., Label-free detection of DNA hybridization using carbon nanotube network field-effect transistor. PNAS, 2006, 103, 921-926. See also, U.S. Patent Application Publication Nos. 2005/0279987 and 2005/129,573. Each Si die containing ten NTFET devices was mounted on a 40-pin standard ceramic dual in-line package (CERDIP).

In several embodiments of a system 10 of the present invention as illustrated in FIG. 1A, a NO to NO₂ converter 20 was used to convert NO to NO₂ via, for example, catalysis. In several studies, chromium trioxide (CrO₃) was used as an oxidizing agent to convert NO to NO₂ in a gas phase. In those studies, approximately 4 g of CrO₃ coated borosilicate glass beads with a diameter of 1 mm were packed in a glass tubing 1 cm in diameter and 10 cm in length. Chromium trioxide (99.98% pure) and 1 mm diameter borosilicate glass beads were purchased from Sigma Aldrich. Ends of the glass tubing were closed with glass wool. The coated beads were prepared by immersion into an aqueous solution of CrO₃ in water (1:5 wt) for 30 min. After filtration on a glass filter, the beads were dried in a vacuum oven at a temperature of 70° C. for two hours.

In several embodiments, NTFET devices of sensors 40 the present invention were made more selective to NO₂ gas by coating the surface of the nanostructures 42 of the chip with a recognition layer such as a poly-ethylenimine (PEI) polymer layer 44. In general, recognition layers (including, for example, one or more polymers having amino functionality) can enhance sensitivity to NO₂. Such polymers include, but are not limited to, one or more of polyethyleneimine (PEI), polyamidoamine (PAMAM), Polydi(carbazol-3-yl)phenylamine, nylon or poly(N-isopropylacylamide) (PNIMAM). In several studies, branched PEI polymer with an average molecular weight of 25000 kDa was used. Water-free branched poly(ethylene imine) (PEI) with a typical Mw 25 000 was obtained from Aldrich, St Louis, Mo. A single drop of 10⁻³ M solution of PEI in tetrahydrofuran (THF) was drop casted on a NTFET device surface. The device was dried for two hours in a fume-hood at ambient temperature. Further baking at, for example, 70° C. can be used to reduce hysteresis in electronic characteristics of the polymer coated NTFET.

Certified NO gas mixtures in nitrogen with concentrations of 1 ppm (0.92 ppm actual) and 10 ppm, a 10% gas mixture of CO₂ in nitrogen, pure nitrogen gas and pure oxygen gas were purchased from National Valley Gases, Inc., of Pennsylvania. Unless otherwise indicated, all percentages of gases set forth herein are set forth as volume percents. As, for example, illustrated in FIG. 2, a combination of three mass flow controllers (MFC) was used to prepare gas mixtures of different concentrations. MFCs (available from UNIT Instruments, Inc. of Yorba Linda, California and MKS, Inc. of Wilmington, Mass.) were electronically controlled as known in the art. Relative humidity (RH) of the gas mixtures was adjusted by passing a stream of nitrogen over a saturated salt solution of LiCl in water (15-30% RH) or pure water (>80% RH). RH and the temperature of the gas mixture were measured using a SENSIRION humidity sensor available from Senserion, Inc. of Westlake Village, California.

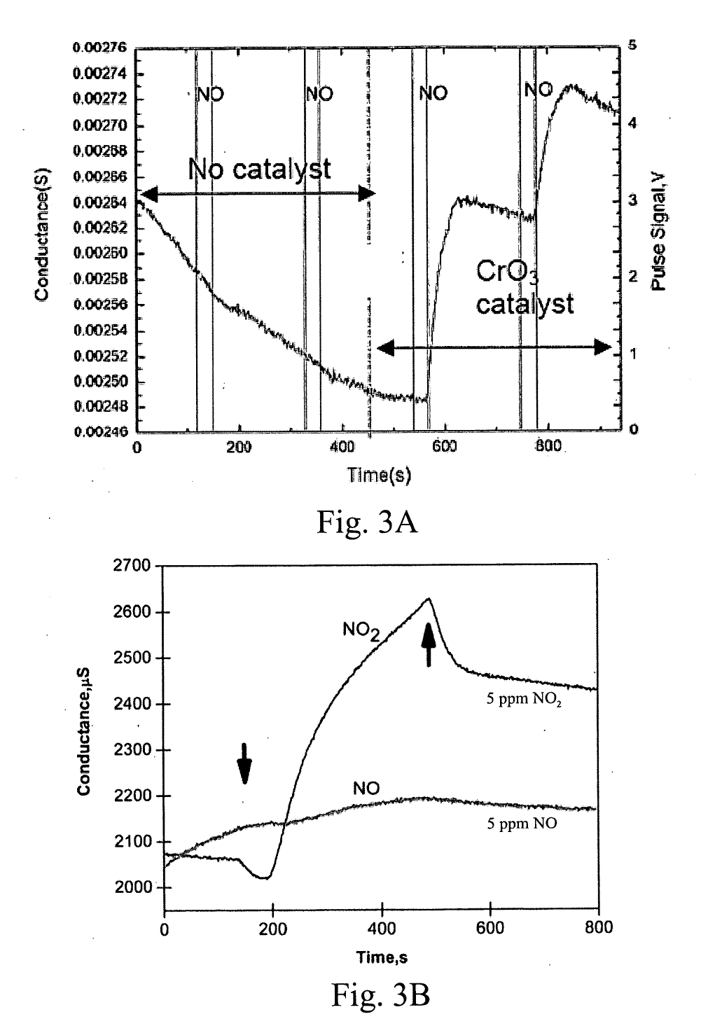

An initial response to NO gas pulses (30 s) passing over bare carbon nanotubes is shown in FIG. 3A. The concentration of NO was approximately 5 ppm. After an initial 500 s, the flow of gas was switched to CrO₃ catalytic converter 20, which created a visible increase in measured conductance. Catalytic conversion was confirmed by experiments with NO and NO₂ gases. A comparison in response between 5 ppm nitrogen dioxide and nitric oxide for a typical, bare NTFET device is shown in FIG. 3B. In FIG. 3B, one trial was performed with 5 ppm NO in N₂. A second trial with 5 ppm NO₂ in N₂ was then performed for comparison to the results with NO. For both trials, the gas was bypassed around the converter 20. The results of those studies suggest that a detection of NO₂ is preferable over pure NO for the sensors tested in those studies as NO may not cause a significant change in conductance at concentration levels of interest. Without limitation to any mechanism, this preference is believed to be a result of the strong electron withdrawing nature of the NO₂ gas versus the weak electron donating nature of NO.

Without proper precautions, a chromium oxide converter can pose a health risk. However, other converters are possible. For example, a noble metal (for example, platinum or a platinum compound) catalyst can be used to convert NO to NO₂ (for example, at elevated temperature). See, for example, Benard, S. et al., Applied Catalysis B: Environmental, 55 (2005) 11-21, Despres, J. et al., Applied Catalysis B: Environmental, 50 (2004) 73-82.

As described above, to improve sensitivity, several NTFET devices of the present invention were functionalized with polymer recognition layers. PEI functionalization of the device surface changes the semiconducting characteristic of the CNFET from p-type to n-type because of electron rich amine groups in the polymer layer (See, for example, FIG. 1B). See Shim M., et al., Polymer Functionalization for Air-Stable n-Type Carbon Nanotube Field-Effect Transistors, J. Am. Chem. Soc. 2001, 123, 11512-11513. The observed hysteresis in the G-V_{G} of curves of FIG. 1C was a result of the flow of humidified N₂.

For a p-type, bare NTFET device, where electron holes are the major carriers, it is expected that the exposure to NO₂, a strong electron withdrawing molecule, will increase the conductance as it is adsorbed on to the SWNT network. On the other hand, an n-type PEI coated NTFET device, with electrons as the major carriers, will show a decrease in conductance when the strong electron withdrawing molecule (NO₂) comes into contact with the PEI-functionalized NTFET. These two trends are observed in comparison of the sensitivities of bare nanotubes and the sensitivities of those functionalized with PEI polymer, as NO is converted to NO₂. Furthermore, because PEI is a nonconductive polymer, conductance is attributed to the SWNT network in contact with the metal electrodes.

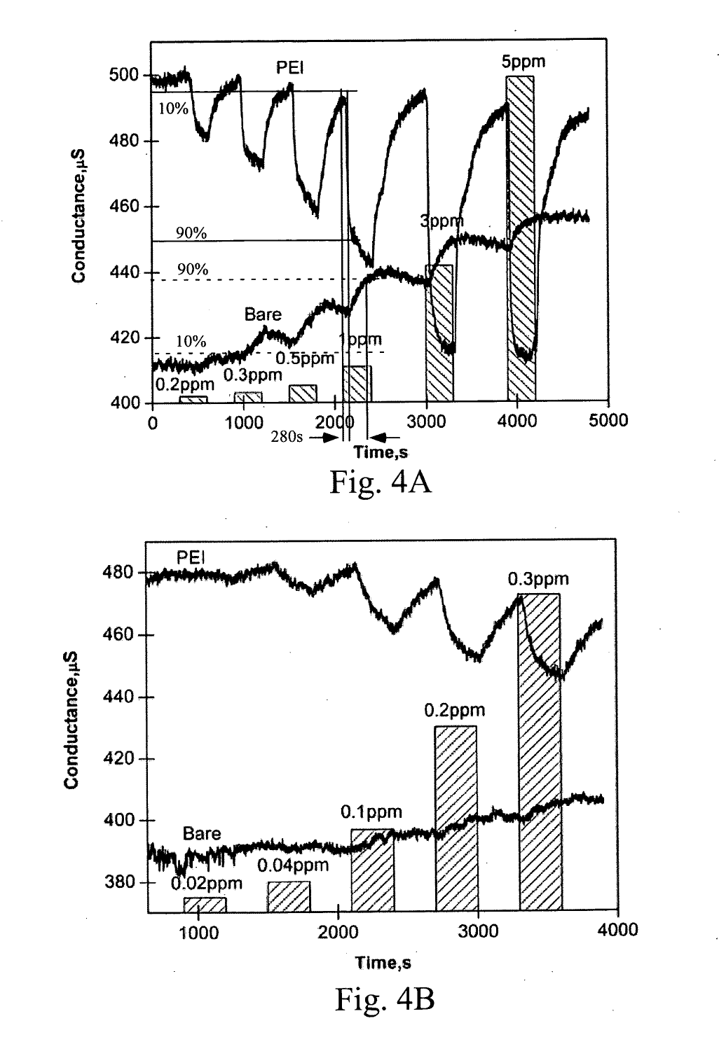

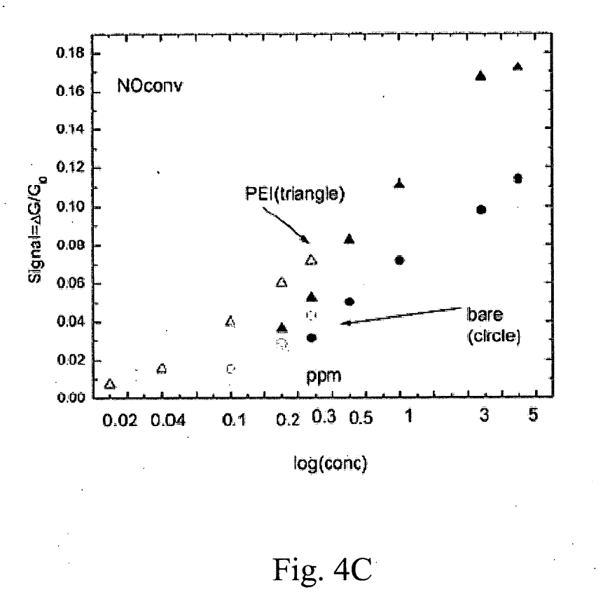

FIGS. 4A and 4B illustrate a decrease in conductance when a PEI-functionalized device is exposed to a series of concentrations of NO gas which were passed through catalyst converter 20 of system 10. In addition, in comparison with a bare device, the sensor response signal was higher and the recovery characteristics were better for the PEI-coated or PEI-functionalized devices. A calibration curve in the concentration range between 20 ppbv and 5 ppmv is set forth for PEI functionalized and bare devices in FIG. 4C. As known in the sensor arts; each NO sensing system of the present invention can undergo an initial and/or periodic calibration to assist in ensuring accurate results.

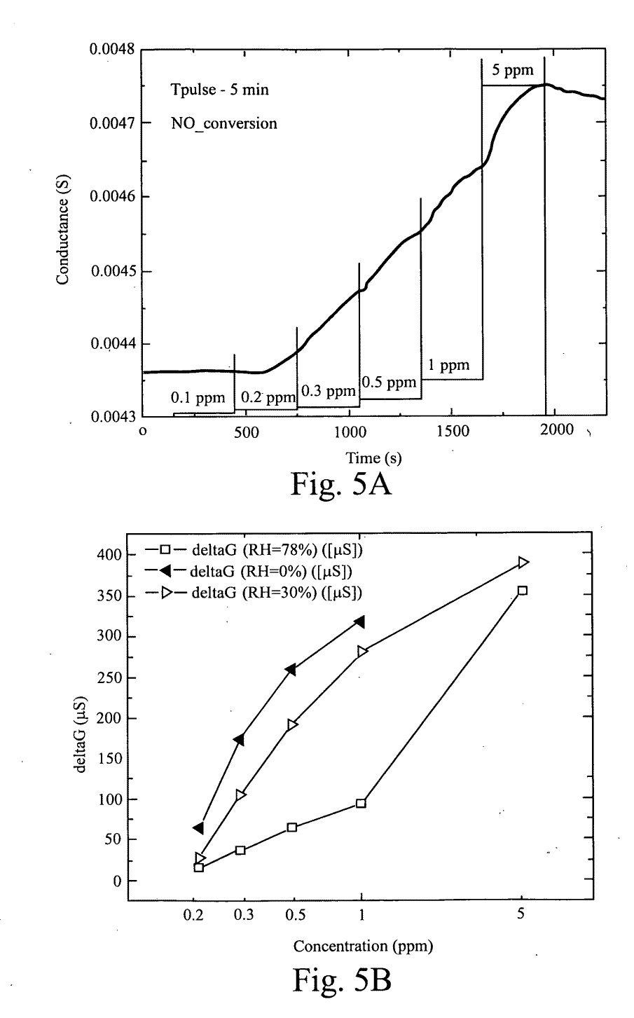

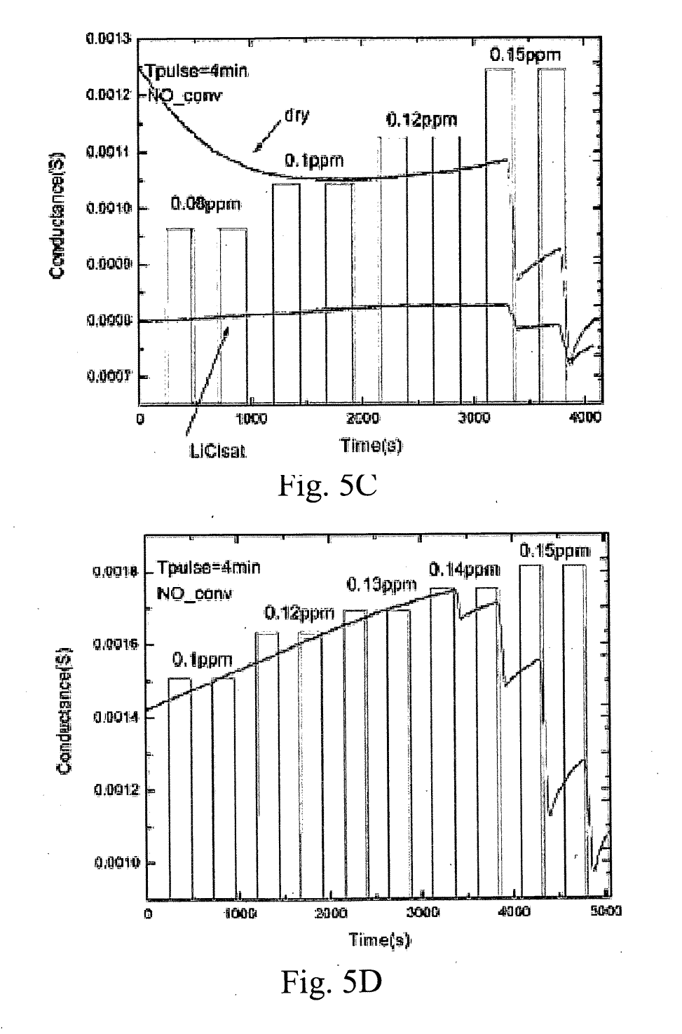

The PEI-coated device showed an overall detection limit around 5 ppbv in an inert atmosphere with 15-30% RH (see FIG. 5A). For the bare device, however, the ultimate detection limit was somewhere between 200 and 300 ppbv. After two weeks of sporadic exposure to the analyte gas, the response to the same concentration of gas decreased. The response time for PEI-coated NTFET devices was much faster than that of bare NTFET devices (in studies of gas mixtures with N₂). The PEI-coated devices exhibited a t₉₀ of approximately 70 s, while the t₉₀ of bare NTFET devices was approximately 280 s. In FIG. 4A, the device baseline conductance for the bare NTFET was 411 μS; while the maximum conductance was 440 μS. The t₉₀ was determined as the time that it took the device to respond from 10% to 90% of this change. A similar t₉₀ calculation was performed for the PEI coated device (with an important difference being a conductance decrease instead of an increase).

The effects of relative humidity (RH) upon the selective detection of NO gas with NTFET devices were also studied. Two potential, interrelated effects of changes in RH on NO detection with NTFET devices were identified. The first effect, f1 (RH), is related to the successful oxidation of NO to NO₂. By use of the CrO₃ converter, humidity acts as an ‘activator’ by adsorbing to the solid. NO then passes across the converter and reacts with the water to become oxidized to the form of NO₂. However, at high RH it is possible for NO to be oxidized to nitrites or nitrates. The second effect, f2 (RH), is directly related to the detection of analyte on carbon nanotubes. NTFETs are known to be sensitive to RH. Therefore, experimentation with various RH concentrations was undertaken to determine optimal conditions for NO detection. The effects of relative humidity on bare and PEI coated NTFET devices were studied. The data of FIGS. 5A through 5D indicate that at any given RH both the magnitude of response and the signal-to-noise ratio is better in PEI coated NTFET devices. The magnitude of signal increases with decreasing RH. By examining the effects of RH humidity, f1 and f2, apparently offset each other. Improved efficiency in the converter occurs at higher RH concentrations, as it provides for a more efficient conversion of NO to NO₂. At lower concentrations, the oxidizing agent dries up and conversion is no longer possible. However, at high concentrations of RH, the effect of f2 hinders the detection of NO by increased hysteresis on the NTFET device. With increased hysteresis, there is an increased level of noise, resulting in a poor signal. A range or approximately 15-30% RH range was identified as a preferred operating range. Increased relative humidity of approximately 100% as found in breath can cause a poor signal in NO detection. However, a number of current medical techniques employ desiccants to decrease the relative humidity in breath during spirometry testing. A desiccant 30 (see FIG. 1A) can, for example, be readily placed before the devices to reduce or to remove residual humidity not conducive to adequate detection of NO.

One proposed mechanism for improved recovery and response time of a PEI coated device versus that of a bare NTFET device involves the interactions taking place on the nanotube surface. This proposed mechanism is contingent upon the effect that RH plays in the exchange acid-base reaction between NO₂ (nitric acid) and PEI. Recovery of bare devices exposed to NO₂ is known to be slow, but can be accelerated at elevated temperatures or by UV illumination. When NO₂ adsorbs to a bare NTFET device, there is a partial withdrawal of electrons from SWNT making it more p-type. The reaction between a PEI coated NTFET device and NO₂, however, results in an acid-base reaction and a change of electron donation of the PEI polymer, thus making it less n-type. The reaction creates an unstable ammonium nitrate salt giving a faster recovery, as NO₂ is not directly adsorbed to the SWNT.

The above mechanism implies that other acids may trigger a false response. A normal person exhales a maximum level of approximately 6% of CO₂ in breath, which is almost seven orders of magnitude higher than the lowest NO concentration in human breath. The chemical reaction between CO₂ and H₂O produces carbonic acid (H₂CO₃), which can also protonate the PEI layer. The formation of carbonic acid is a reversible reaction that readily depends upon water concentration, (that is, relative humidity). Although carbonic acid is a weak acid (pKa=3.6) compared to nitric acid (pKa=−1.3), a large excess in CO₂ concentration compared to NO₂ may cause a significant interference.

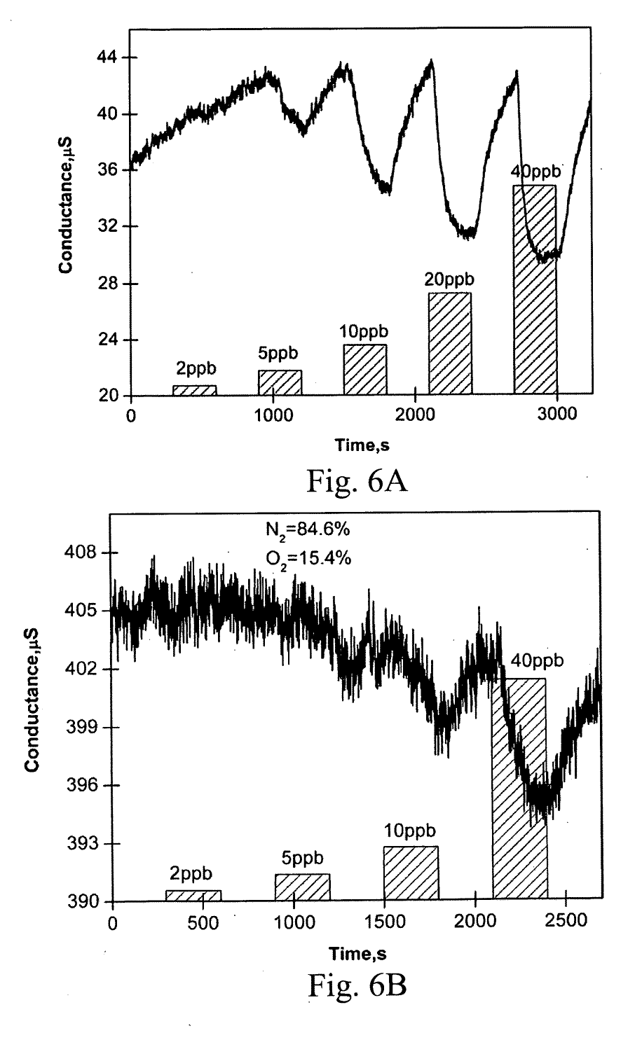

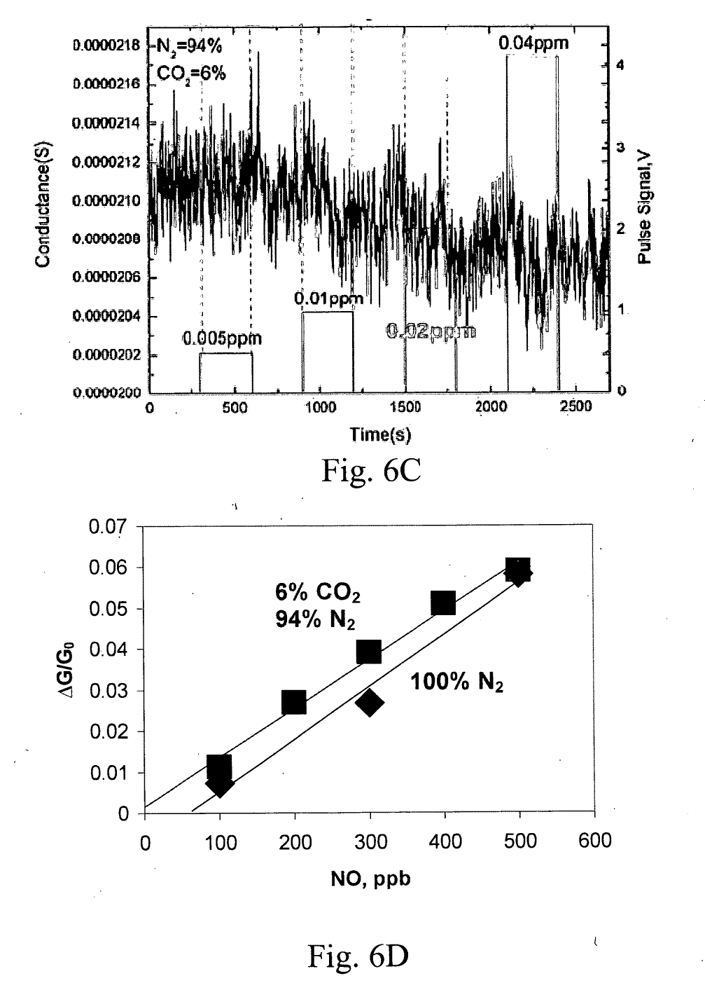

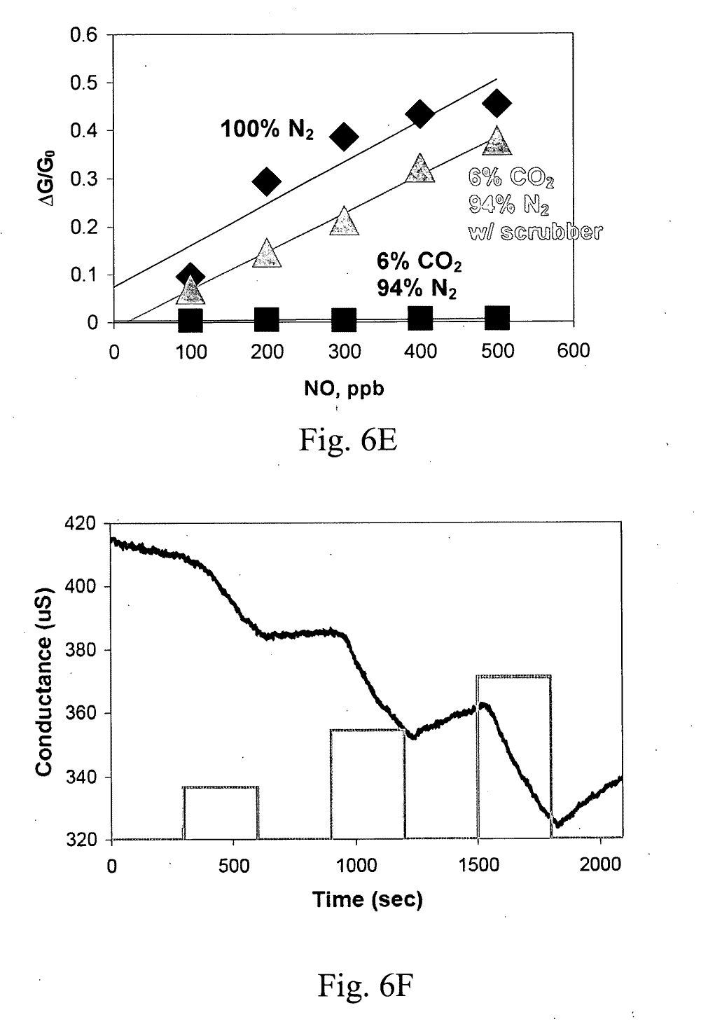

To further simulate conditions similar to that of breath, the cross-sensitivities of major components found in breath such as CO₂ and O₂ were also studied. FIGS. 6A, 6B and 6C illustrate conductance versus time dependence of a PEI coated NTFET device of the present invention exposed to five short pulses of NO gas at concentration range between 2 PPB and 60 PPB in a 100% nitrogen atmosphere, a 15.4% oxygen atmosphere balanced with nitrogen, and a 6% carbon dioxide atmosphere balanced with nitrogen, respectively.

Oxygen, (15.4% volume) does not seem to interfere with the detection of analyte gas in the desired concentration range (see FIG. 6B). In the studies of FIG. 6B, a PEI-coated NTFET device was tested for NO at a concentration range of 2-40 ppb in 15.4% O₂ and 84.6% N₂ gas mixture. Converted NO was detected noticeably from 10 ppb and above. As the lower end of the desired detection region for human breath is approximately 20 ppb for NO, the presence of O₂ does not significantly affect the detection capability.

Results of carbon dioxide cross-sensitivity studies (FIG. 6C) show that a PEI coated device is cross-sensitive to CO₂ in the range of approximately 1-6%. In the atmosphere of 6% CO₂, detection of low concentrations of NO converted into NO₂ becomes very difficult. As illustrated in FIG. 1A, a filtering device such as a scrubber 60 for CO₂ removal can be used in system 10 of the present invention to remove the CO₂ interferent. ASCARITE® (a registered trademark of Arthur H. Thomas, Co. DBA Thomas Scientific Corporation of Swedesboro, New Jersey, which is a sodium hydroxide-coated silica adsorbent) was used in CO₂ scrubber 60 and was purchased from Sigma-Aldrich. CO₂ scrubber 60 was fabricated from a quartz J-tube containing 5.0 g of ASCARITE with glass wool and parafilm creating a leak-free seal. A gas inlet and outlet was exposed on either end of the quartz J-tube to allow connection of tubing carrying the analyte gas mixture.

Unlike PEI-coated devices, it was found that the bare devices did not respond to the same concentrations of CO₂ (data not shown). FIGS. 6D and 6E illustrate calibration plots of bare and PEI coated NTFET devices. In FIG. 6E, the slopes, correlating to sensitivity, for the bare NTFET device in both 100% N₂ and 94% N₂ with 6% CO₂ are approximately the same, indicating a minimal effect of CO₂ on device sensitivity. Also, as seen from the calibration plots, the slope of the curve for bare NTFET devices indicates a better sensitivity than that of PEI coated NTFET devices in an environment of 94% N₂ and 6% CO₂. On bare NTFET devices, it is still possible to achieve detection of converted NO. At 200 ppb, there is a defined signal, indicating electron withdrawal and increasing conductance. The data of FIG. 6E indicate that PEI and CO₂ react to alter the initial state of the PEI polymer. This reaction creates an extensive amount of noise and shows up as an almost random assignment through the R2 value in the calibration plot. As discussed above and without limitation to any mechanism, it has been hypothesized that the reaction between the amine groups of PEI and CO₂ gas form carbamates, thus modifying the polymer. At a concentration of 6% CO₂ by volume, this effect is evidenced by the different response achieved with a bare NTFET device and converted NO. The interference with PEI-coated devices created by CO₂ (and/or other interferents such as acidic gases) can be substantially reduced or eliminated using scrubber 60 to remove CO₂ (and/or other interferents such as acid gasses).

By placing filtering device/scrubber 60 (a quartz J-tube with an excess of 5.0 g of ASCARITE as described above) before oxidizing agent 20 (see FIG. 2), an appreciable amount of CO₂ and NO₂ were removed, thereby allowing detection of NO. Unlike NO₂ and CO₂, NO is not acidic and is able to continue through scrubber 60 and to converter 20, where it is oxidized to NO₂. The results indicate that a negligible amount of NO, if any, was absorbed on scrubber 60. Thus, when conducting a human breath sample, it should be possible to monitor NO as it is converted to NO₂. Exhaled NO₂ or CO₂ will be trapped in scrubber 60, prior to NO arriving at converter 60 (and, subsequently, at NTFET device 40). For this reason, it was preferable to place scrubber 60 before converter 20. Several studies summarized in FIG. 6F demonstrated detection of a signal for NO at 100-500 ppb in a gas mixture of 94% N₂ and 6% CO₂. The capacity of scrubbing shows a detection limit of approximately 15 ppb upon extrapolation of the linear trend between 100 and 500 ppb, which is in the range of detection for lower NO levels in exhaled breath of a healthy patient. The trapping of acidic gases was not optimized for efficiency in the studies of the present invention.

The above studies illustrate the detection of NO using chemically functionalized carbon nanotubes with a high degree of sensitivity and selectivity towards NO/NO₂ over oxygen and water. In several studied embodiments, inclusion of a filter device or scrubber for CO₂/acid gas removal (and/or other acid gas removal) in the systems of the present invention enabled the systems to meet diagnostic requirements for a human breath asthma sensor. Use of a suitable recognition layer that enhances sensitivity to NO₂ without exhibiting substantial cross-sensitivity to CO₂ (and/or, for example, other acid gases) can eliminate the need for such a scrubber. Further, deposition of a suitable catalyst upon an NTFET device can eliminate the requirement of a separate NO to NO₂ converter. Still further, suitable recognitions layers may allow direct measurement of NO gas without conversion thereof to NO₂.

For example, in a number of studies NTFET devices were decorated with metal nanoparticles via electrochemical deposition using a CH Instruments electrochemical analyzer by connecting the source and drain pins of a single device and using it as the working electrode in an electrochemical cell. An epoxy coating (Epoxy Technologies) was used to isolate device leads from the rest of the electrochemical cell to ensure metal deposition only on individual device surfaces. A single drop (approximately 100 μL) of 1 mM H₂PtCl₆, HAuCl₄, AgNO₃, or 0.5 mM K₂PdCl₆ (Sigma Aldrich) in a supporting electrolyte of 0.1 M HCl or KNO₃ (for AgNO₃) was placed on the NTFET chip with Ag/AgCl reference and Pt wire counter electrodes just in contact with the surface of the solution to create a miniaturized electrochemical cell. A deposition potential of −1.0 V was held for a time between 10 and 90 s to deposit metal nanoparticles of various sizes on the device SWNT networks. Scanning electron microscopy (SEM) was performed with a Phillips XL30 FEG microscope equipped with an EDAX assembly for energy dispersive spectroscopy (EDS) allowing confirmation of metal deposition. For experiments, chips were tested using an NTFET electronic test fixture such as described in Star, A.; Joshi, V.; Skarupo, S.; Thomas, D.; Gabriel, J.-C. P. J. Phys. Chem. B 2006, 110, 21014, wherein research grade N₂ and 10.0 ppm NO gas in N₂ (Valley National Gas) were passed over the metal-decorated chips and conductance versus gate voltage (G-V_{G}) transfer characteristics of all devices were simultaneously recorded at room temperature.

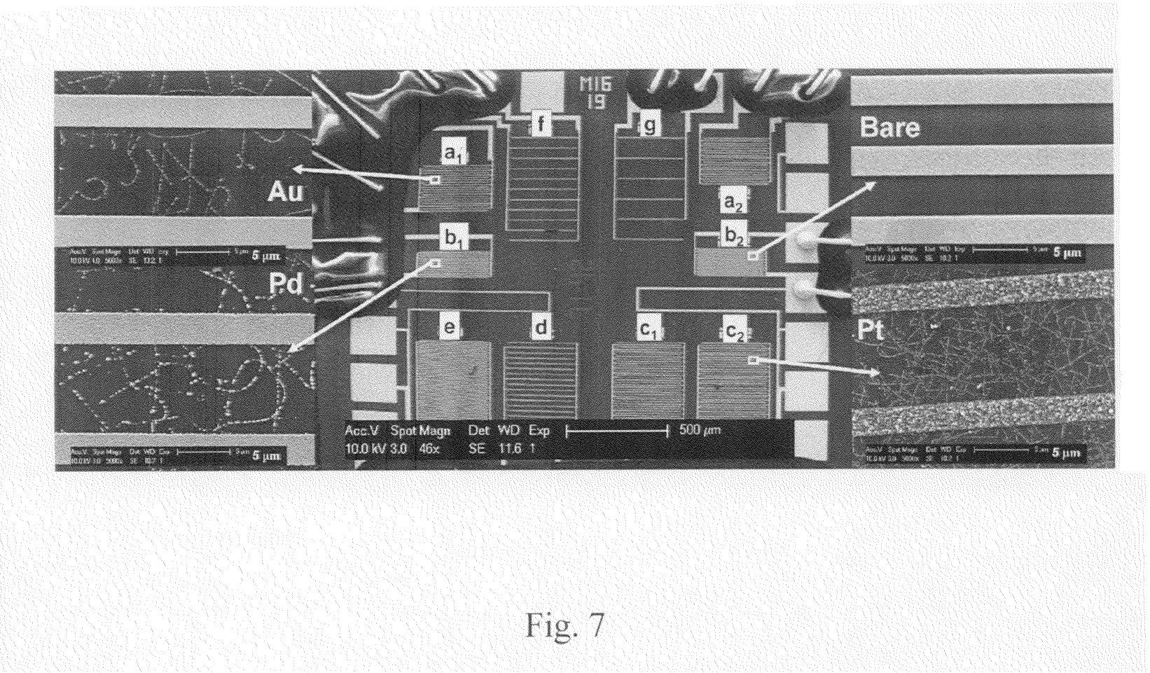

The NTFET chips used in these studies contained multiple devices on the surface, as shown in the center of FIG. 7, wherein the epoxy coating (black) is seen surrounding the wirebonded contacts. Three sets of degenerate devices, each with identical geometry and electrode separation (pitch), were present on each chip. Degenerate devices are denoted with subscripts. Through the selective electrochemical deposition of metals, it was possible to decorate particular devices while leaving their corresponding degenerate devices bare. To demonstrate selective decoration, a deposition time of 60 s was used to deposit approximately 150-300 nm Au nanoparticles on device a₁, 90 s was used to deposit approximately 150 nm Pd nanoparticles on device b₁, and 30 s was used to deposit approximately 120 nm Pt nanoparticles on device c₂ while their complementary degenerate devices were left bare. Longer deposition times resulted in a nearly complete loss of NTFET transfer characteristics as a result of large nanoparticles screening the gate voltage. To reduce or eliminate this effect, smaller deposition times of between 10 and 20 s were used to decorate chips for gas exposure experiments.

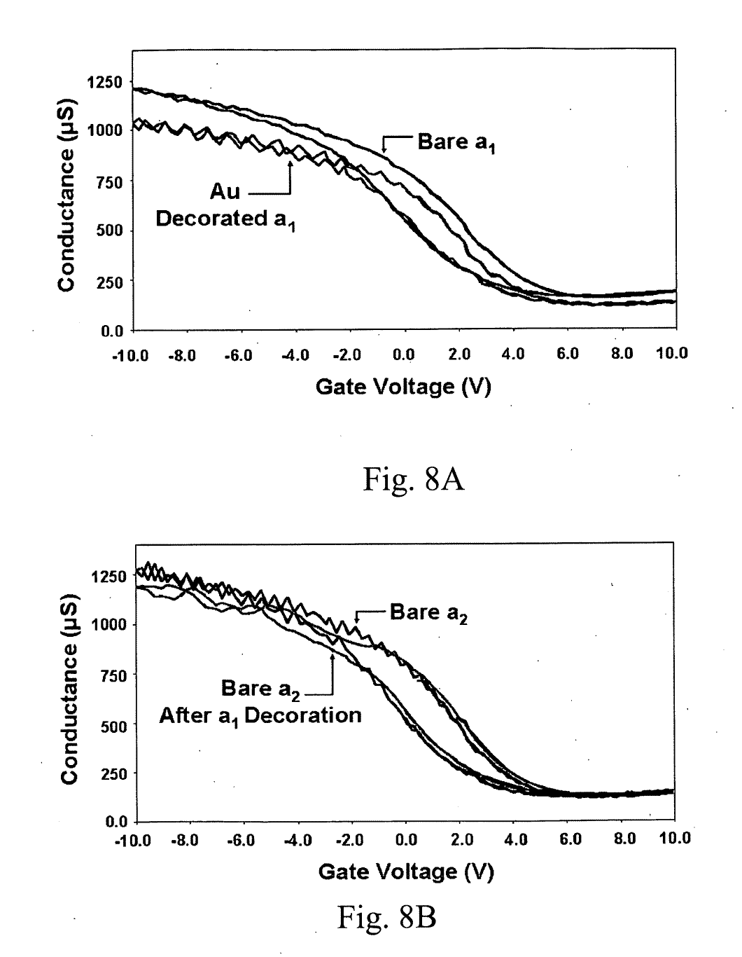

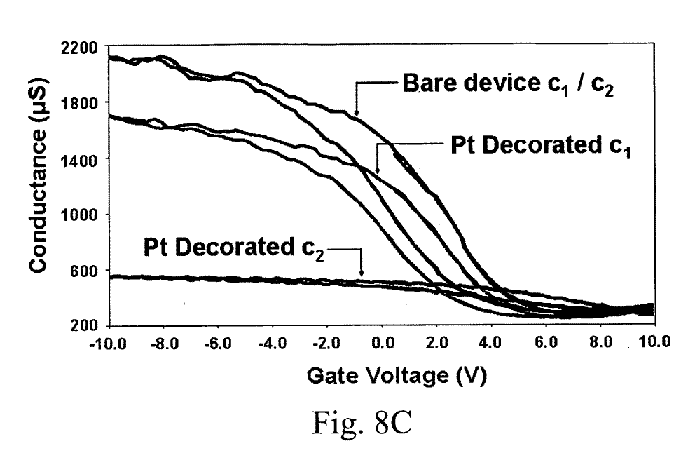

FIGS. 8A and 8B show the G-V_{G} transfer characteristics of degenerate devices a₁ and a₂ from a first (chip 1) of two chips. During deposition, both devices were in the HAuCl₄/HCl solution. FIG. 8A shows that after device a₁ was held at potential for 20 s it experienced modest G-V_{G} modification. FIG. 8B, however, shows that the G-V_{G} transfer characteristic of device a₂, which was not held at potential, remained essentially unchanged. This result indicates Au nanoparticles were selectively deposited on device a₁, while a₂ remained pristine. FIG. 8C shows the modification of chip 1 devices c₁ and c₂ transfer characteristics resulting from different Pt deposition times. Both devices were independently held in the H₂PtCl₆/HCl solution at −1.0 V for different amounts of time. Device c₂ was held at potential for 20 s, while device c₁ was held at potential for only 10 s to obtain a smaller Pt coverage on the SWNT surface. It can be seen that while device c₁ experienced modest G-V_{G} modification, device c₂ experienced an almost complete loss of gate voltage dependency as a result of increased Pt deposition time. Subsequent SEM images showed small Pt nanoparticles approximately 20 nm in diameter in device c₁ and comparatively large 50-100 nm diameter particles in device c₂. These results are representative of the G-V_{G} modifications seen in devices on both chips upon electrodecoration

G-V_{G} transfer characteristics of all devices on a particular NTFET chip were simultaneously monitored under a 300 SCCM flow of dry N₂ and 10 ppm NO in N₂. The flow system was flushed with dry N₂ for 1 h prior to NO exposure to remove any O₂ or humidity present from atmospheric exposure during insertion of the NTFET chip. Removal of O₂ and trace H₂O from the flow system was necessary to ensure NO did not undergo any Red/Ox reactions in transit to the chip. Initially NO exposure caused a positive shift in device gate voltage which reversed and stabilized upon further exposure. Without limitation to any mechanism, it was hypothesized that NO consumed surface-bound oxygen species on the metal nanoparticle and, by producing electron-withdrawing NO₂, caused the positive shift in device gate voltage. Further NO exposure depleted the surface-bound oxygen and eventually resulted in a NO-saturated equilibrium on the nanoparticle surface. The duration of the positive shift in device gate voltage associated with NO₂ production varied depending on the metal species, but an exposure time of 1 h was used for all experiments to ensure measurement in a homogeneous atmosphere and equilibrium state NO coverage on the nanoparticle. As NO has an unpaired electron it is expected to be a weak electron donor, and after the device transfer characteristics stabilized, it was found that exposure to 10 ppm NO resulted in a small decrease in conductance and negative shift in gate voltage for all devices on the same chip, with metal-decorated devices showing consistently larger gate voltage shifts.

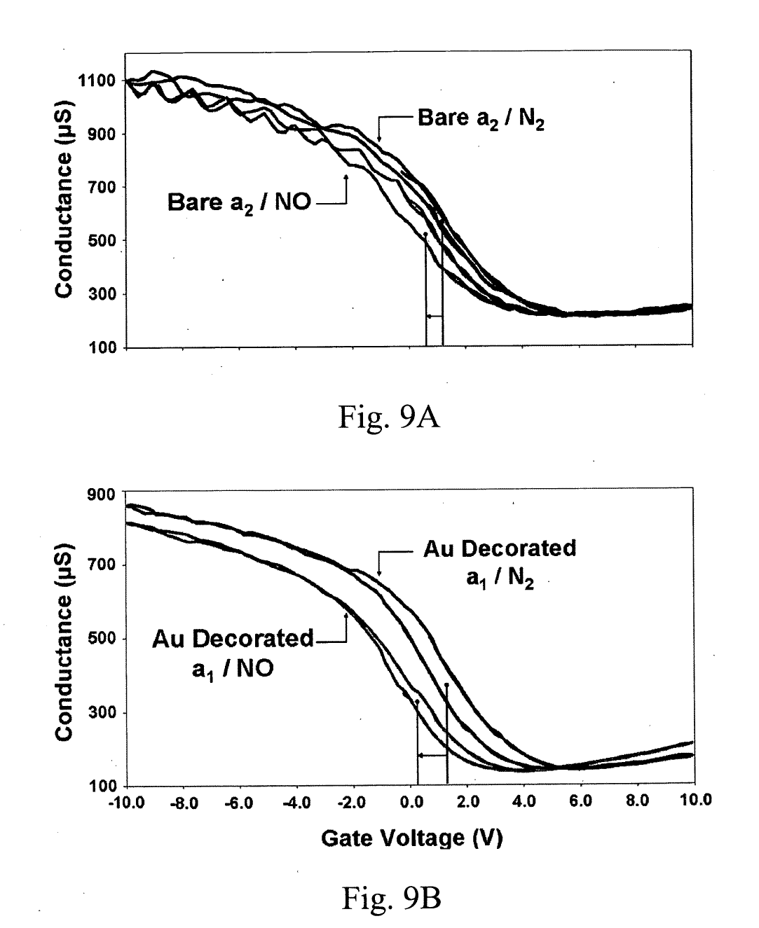

FIGS. 9A and 9B, respectively, show the representative response of chip 1 devices a₂ (bare) and a₁ (Au-decorated) upon exposure to dry 10 ppm NO in N₂. NO exposure caused a smaller gate voltage shift in the bare device a₂ (FIG. 9A) compared to the Au-decorated device a₁ (FIG. 9B), indicating increased electronic donation into the SWNT network of device a₁. The deposited metal nanoparticles and bulk NTFET contacts are both composed of Au which indicated that the increased gate voltage shift in the Au nanoparticle decorated device is a result of an NO-nanoparticle interaction. The G-V_{G} transfer characteristics of six bare devices and three metal-decorated devices were monitored on the two NTFET chips during gas exposure. For a particular chip it was found that bare devices with different geometries and electrode separations (pitch) showed consistent gate voltage shifts, and metal decorated devices all demonstrated unique gate voltage shifts larger than the bare devices. Additionally, the gate voltage shifts in metal-decorated devices showed a dependency on the metal work function, wherein larger shifts were seen in devices decorated with metals having smaller work function. Although the two NTFET chips (chip 1 and chip 2) demonstrated different magnitude responses to NO gas, the trend between smaller work function and increased device gate voltage shift was consistent.

After comparing degenerate bare and decorated devices on separate chips, and finding the trend was independent of the particular device geometry, all four metals were used to decorate devices on a single chip (chip 3). Two devices on chip 3 were decorated with each metal for either 10 or 20 s as set forth in Table 1 below. Depositing the metals in this manner allowed a direct comparison between metal-decorated NTFET devices on one chip, removing any inconsistency in response magnitude between individual chips. Additionally, this allowed the comparison between the metal nanoparticle size and device response to be made for each metal. It was found that devices decorated with a particular metal nanoparticle showed equivalent gate voltage shifts upon NO exposure regardless of the difference in particle size, which indicates that metal work function, and not particle size, influenced electronic donation into the SWNT network.

TABLE 1

| | | | | | work | |
|---|---|---|---|---|---|---|
| | pitch | | deposition | particle | function | gate voltage |
| device | (μm) | metal | time (s) | size (nm) | (eV) | shift (V) |
| all | 5-100 | bare | 0 | N/A | N/A | −0.30 ± 0.02 |
| a₁ | 10 | Ag | 10 | 70-140 | 4.26 | −0.84 |
| a₂ | 10 | Ag | 20 | 80-200 | 4.26 | −0.83 |
| b₁ | 5 | Au | 10 | 41-82 | ~5.0 | −0.75 |
| b₂ | 5 | Au | 20 | 113-148 | ~5.0 | −0.73 |
| e | 5 | Pd | 10 | 45-80 | 5.12 | −0.66 |
| d | 25 | Pd | 20 | 100-190 | 5.12 | −0.69 |
| c₁ | 10 | Pt | 10 | 40-80 | 5.65 | −0.57 |
| c₂ | 10 | Pt | 20 | 80-200 | 5.65 | −0.55 |

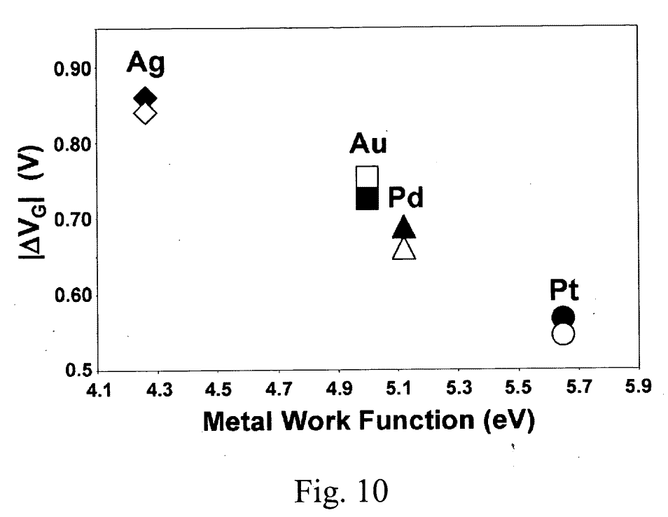

Each metal-decorated NTFET device had a unique gate voltage shift upon exposure to NO gas, and when the absolute value of the shift was plotted against the metal work function (Φ), a clear trend was found, wherein a smaller work function led to a larger gate voltage shift. FIG. 10 shows the relationship between metal work function and absolute value of the voltage shifts in response to NO for metal-decorated devices on chip 3, wherein hollow and solid labels represent the response of devices held at a deposition time of 10 (solid labels) or 20 s (hollow labels). It is noted that there is some discrepancy found in literature values for Φ_{Au}. Common values for Φ_{Pd}, Φ_{Pt}, and Φ_{Ag} were used, and in the absence of a consistent value for (Φ_{Au}, a value of approximately 5.0 eV was chosen. In general, use of any reasonable literature value for Φ_{Au} would still show a similar trend to that set forth in FIG. 10.

As described above, the sensors of the present invention use a change in the conductivity of nanotubes to detect, for example, NO or NO₂ (which can, for example, be produced by conversion of NO in exhaled breath to NO₂). Compared to previous sensors used to detect relatively low concentrations of NO and/or NO₂ in, for example, medical applications (such as asthma diagnosis), the nanotube based sensors and systems of the present invention are much smaller (on the order of microns), much less expensive to produce and simpler to operate. Such qualities in the devices, systems and methods of the present invention enable disposal of the sensor or sensor system on, for example, a per-use or per-patient basis.

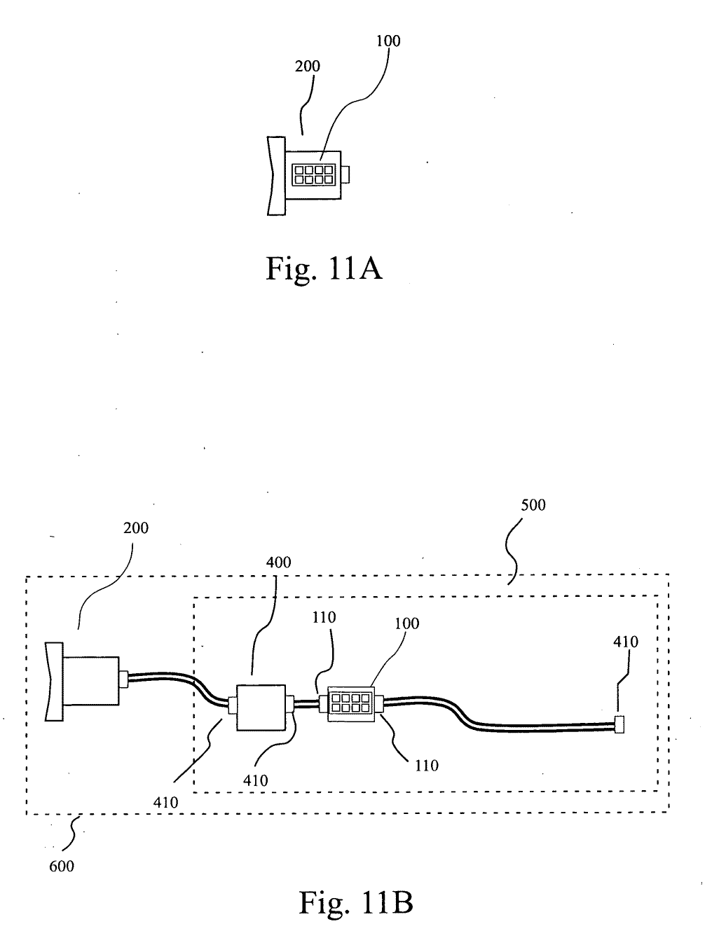

As for example, illustrated in FIG. 11A a disposable sensor system 100 of the present invention can be incorporated into or associated with a handheld mouthpiece or mask 200 into which a patient exhales. Other elements such as one or more filters/scrubbers, one or more converters, on or more flow meters etc. can also be incorporated into or associated with handheld mouthpiece or mask 200. Additionally or alternatively, a sensor system of the present invention can be attached to mouthpiece or mask 200 or to a flow path 210 (for example, flexible tubing) attached to mouthpiece or mask 200 (see FIG. 6B). Sensor system 100 can, for example, include an NO and/or NO₂ sensor as described above. Further sensors for other exhalation gases that operate based on changes in nanoelectrical properties (for example, conductivity) of nanostructures/nanotubes can also be included in sensor system 100. For example, nanostructure sensors can be included to enable the sensing of CO₂ and/or CO (the concentration of which, in addition to the concentration of NO, can be useful, for example, in diagnosis of or characterization of asthma). Sensor arrays comprising a plurality of carbon nanotube sensors can be used in the present invention. Sensor arrays and the processing of data therefrom are described, for example, in Qui, P. et al., Toward large Arrays of multiplex functionalized carbon nanotube sensors for highly sensitive and selective molecular detection, Nano Lett. 2003, 3, 347-351 and Star, A. et al., Gas sensor array based on metal-decorated carbon nanotubes, J. Phys. Chem. B 2006, 110, 21014-21020. Use of sensor arrays including a plurality of different sensors may, for example, enable detection of various gasses even in the presence of interferrent gases (that is, gases to which a particular sensor exhibits cross-sensitivity). As described above, problems with cross-sensitivity can also be reduced or eliminated with the use of one or more filter devices or scrubbers in one or multiple of flow paths (into which flow can be split).

A sensor or sensor system 100 of the present invention can, for example, include a connector 110 (see FIG. 11B) thereon (for example, Luer connectors as known in the art) to readily enable connection there of to a fluid path or fluid path element (for example, to a flow tube or to mouthpiece or mask 200). Other elements, including, for example, one or more converters (for example, an NO to NO₂ converter), one or more scrubbers (to, for example, interferents such as CO₂), one or more flow meters or sensors and/or one or more pressure sensors (represented collectively in FIG. 11B by element 400) can also be readily and removably connectible to a fluid path or fluid path element via connectors 410 (for example, Luer connectors). Flow meters or flow rate sensors and other types of sensors such as pressure sensors, volume sensor etc. can, for example, be made to be relatively small and inexpensive. Such sensors can, for example, be fabricated using MEMS or lab-on-a-chip technologies as known in the art. A primary commercial source for lab-on-a-chip technology is, for example, ISSY-Integrated Sensing Systems, Inc. of Ypsilanti, Michigan.

Various nanostructure gas sensors, filter devices/scrubbers, converters and/or other sensors as described above can, for example, be provided as a unit or system 500 (illustrated within dashed lines in FIG. 11B). System 500 can, for example, packaged and distributed to be removably attachable to a fluid path or fluid path element via connectors 410 as described above. Mouthpiece or mask 200 can also be provided as part of a system 600 as illustrated in FIG. 11B. Entire system 500 or system 600 (or components thereof) can, for example, be disposable on, for example, a per-use or per-patient basis.

Typically, in an asthma diagnosis and/or characterization a peak flow meter is used. A peak flow meter is a simple, portable and inexpensive device which measures air flow, or peak expiratory flow rate (PEFR). Peak expiratory flow rate is the maximum volume rate of air one can blow during the first portion (for example, the first second of first seconds) of expiration. Peak flow meter can help to determine how open airways are. See, Booras, C. H., Peak Flow Meter—A Thermometer for Asthma, 1998 (available at www.jaxmed.com/articles/Diseases/peakflow.htm). Peak flow meters can be useful, for example, to determine the severity of asthma, check the response to treatment during an asthma episode, monitor progress in treatment, detect worsening in lung function and diagnose exercise-induced asthma. The flow meters and/or volume meters described above can be a peak flow meter. Moreover, sensor systems of the present invention can, for example, be incorporated in or associated with a peak flow meter.

Asthma sufferers, to date, are diagnosed by means of a variety of tests that either need to induce an asthmatic attack, rely on bulky equipment, or simply do not work in a real time format. The devices, systems and methods of the present invention can provide real time measurements, portability, and biomarker detection not previously attainable. The devices, systems and methods of the present invention can be utilized in reliable medical equipment for use in settings ranging from hospitals to disposable home diagnostics care for in-house treatment.

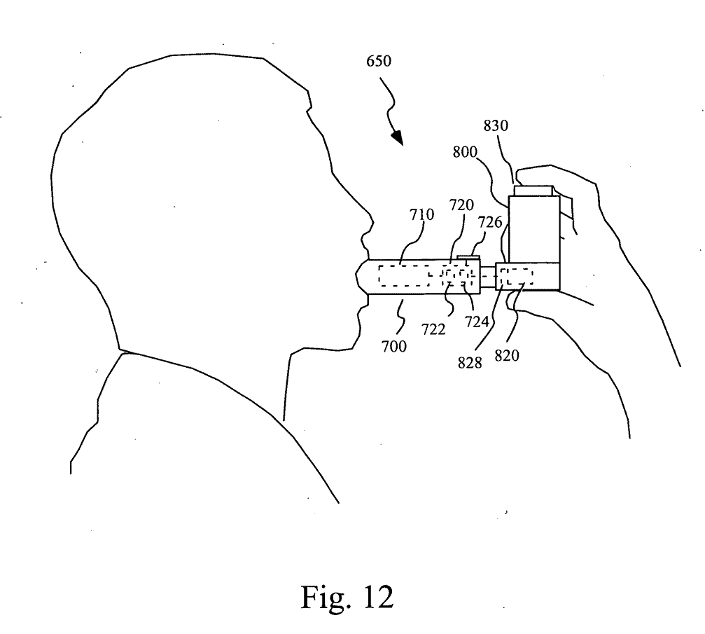

As illustrated in FIG. 12, the sensing systems of the present invention can, for example, be used as a part of inhaler or inhalator system 650 wherein measurement of NO concentration in breath as described above is used, for example, to determine administration of treatment/rescue asthma medication and/or to determine if preventative/controller asthma medication is required (and/or a dose therefor). In that regard, asthma medicines can generally be divided into two groups: (1) medicines to prevent attacks (sometimes referred to as controller medicines), and (2) medicines to treat attacks (sometimes referred to as rescue medicines).

Inhaler system 650 can, for example, include a mouthpiece or spacer system 700 attachable to, for example, a metered-dose inhaler 800. Mouthpiece system 700 includes a sensor system 710 (for example, similar to sensor system 10 or sensor system 100) as described above to detect NO directly or via conversion to NO₂. Although discussed herein as a removably attachable mouthpiece system 700, mouthpiece system 700 can be integrally or nonremovably connected to inhaler 800 as the mouthpiece therefor. The relatively small size of the systems of the present invention enable the incorporation of the components thereof within inhaler 800 or within a mouthpiece therefor.

Mouthpiece 700 can also include a controller 720 (for example, including a microprocessor 722 and an associated memory 724) in communicative connection with sensor system 710. All or any portion of mouthpiece 700 can be disposable (or reusable). For example, controller 720 can be removably connectible and reusable while other components are disposable. Controller 720 can, for example, be in communicative connection with a display 730 to provide information related to a detected concentration or NO to the user. The information can also, for example, include information of a proper dose to be distributed from metered-dose inhaler 800. The user can, for example, use this information to adjust the dose delivered by inhaler 800. Controller 720 can also be in operative connection with a control system 820 (for example, including a microprocessor and an associated memory) of metered-dose inhaler 800. Controller 820 can, for example, include or be in operative connection with a metering valve 828, as known in the art, to effect control the dose delivered from inhaler 800. Each of controllers 720 and 820 can be battery (not shown) powered as known in the art. A signal from controller 720 related to a determined level of NO in the user's breath can be transmitted to controller 820 so that controller 820 can effect a proper dose from inhaler 800 upon user actuation of actuator 830. Alternatively, controller 720 can directly control the dose delivered by inhaler 800.

During use of system 650, the user can, for example, breath into mouthpiece 700 while disconnected from inhaler 800 to measure the concentration of NO in the user's breath. Mouthpiece 700 can then, for example, be attached to inhaler 800 to deliver medication as needed.

Mouthpiece 700 can also be used periodically (for example, daily) as a standalone unit as described above to determine if there is an elevated level of NO in the user's breath to determine whether (and/or in what amount) a dose of controller medicine is required. Studies have shown that elevated levels of NO in breath precede asthma attack for some time (for example, days). Currently, patients with severe asthma may take preventative or controller medicine each day to prevent an attack, very often resulting in overmedication. Others may fail to take controller medicine when warranted. Mouthpiece 700 can, for example, be used to determine whether controller medicines should be taken and/or to determine an appropriate dose therefor. The devices, systems and methods of the present invention can thus be used to effect appropriate dosing of controller medications and rescue medications for asthma sufferers.

The foregoing description and accompanying drawings set forth the preferred embodiments of the invention at the present time. Various modifications, additions and alternative designs will, of course, become apparent to those skilled in the art in light of the foregoing teachings without departing from the scope of the invention. The scope of the invention is indicated by the following claims rather than by the foregoing description. All changes and variations that fall within the meaning and range of equivalency of the claims are to be embraced within their scope.

What is claimed is:

1. A system for the detection of nitric oxide in a gas sample comprising:
a converter for oxidation of nitric oxide to nitrogen dioxide;
a nitrogen dioxide sensor comprising at least one nanostructure; and
a filtering device to remove at least carbon dioxide from the gas sample positioned upstream of the converter.

2. The system of claim 1 wherein the nitrogen dioxide sensor comprises a recognition layer in contact with the at least one nanostructure to enhance sensitivity to nitrogen dioxide.

3. The system of claim 2 wherein the recognition layer comprises at least one polymer, a metal or a metal compound.

4. The system of claim 2 wherein the recognition layer comprises a polymer including amino functionality.

5. The system of claim 2 wherein the recognition layer comprises at least one of polyethyleneimine (PEI), polyamidoamine (PAMAM), Polydi(carbazol-3-yl)phenylamine, nylon or poly(N-isopropylacylamide) (PNIMAM).

6. The system of claim 2 wherein the recognition layer comprises polyethyleneimine.

7. The system of claim 1 wherein the filtering device is operable to remove acid gases.

8. The system of claim 7 wherein the filtering device comprises an acid gas adsorbent.

9. The system of claim 1 wherein the converter comprises chromium trioxide.

10. The system of claim 1 further comprising at least one other sensor comprising nanostructures to sense a gas other than nitrogen dioxide.

11. The system of claim 1 further comprising a plurality of other sensors comprising nanostructures to sense gases other than nitrogen dioxide.

12. The system of claim 10 wherein the other sensor comprises a carbon monoxide sensor or a carbon dioxide sensor.

13. The system of claim 1 further comprising a flow meter to measure rate of flow.

14. The system of claim 13 wherein the flow meter is a peak flow meter.

15. The system of claim 14 wherein peak flow meter is adapted to measure rate of flow and volume of exhaled gas.

16. The system of claim 1 further comprising a sensor to measure volume of exhaled gas.

17. The system of claim 1 wherein at least the nitrogen dioxide sensor is disposable.

18. The system of claim 1 wherein the nitrogen dioxide sensor is in communicative connection with a controller, the controller being adapted to determine a level of nitric oxide at least in part on a basis of output from the nitrogen dioxide sensor.

19. The system of claim 18 wherein the controller is in communicative connection with a display to display information related to a determined level of nitric oxide.

20. The system of claim 18 further comprising a mouthpiece system into which a user can exhale in fluid connection with the converter and the nitrogen dioxide sensor.

21. The system of claim 19 wherein the mouthpiece is in connection with a medication dosing system.

22. The system of claim 19 wherein the mouthpiece is removably connectible to a medication dosing system.

23. The system of claim 21 wherein the controller provides information to the medication dosing system related to the determined level of nitric oxide.

24. The system of claim 20 wherein the converter and the nitrogen dioxide sensor are components of the mouthpiece system.

25. The system of claim 23 wherein the controller is a component of the mouthpiece system.

26. The system of claim 1 wherein the filtering device, the converter, and the nitrogen dioxide are components of a medication dosing system.

27. The system of claim 26 wherein the medication dosing system is an inhaler.

28. The system of claim 1 wherein the nitrogen dioxide sensor comprises a network of carbon nanotubes.

29. The system of claim 28 wherein a source electrode is in contact with the network of carbon nanotubes and a drain electrode is in contact with the network of carbon nanotubes.

30. The system of claim 1 wherein the nitrogen dioxide sensor comprises a nanotube field effect transistor (NTFET) device.

31. The system of claim 1 further comprising a system to reduce relative humidity of the gas sample prior to contact with the nanostructures.

32. A method for detecting nitric oxide in a gas sample comprising:
filtering the gas sample to remove at least carbon dioxide from the gas sample;
after filtering the gas sample, oxidizing nitric oxide in the gas sample to nitrogen dioxide; and
detecting the nitrogen dioxide using a nitrogen dioxide sensor comprising at least one nanostructure.

33. The method of claim 32 wherein the gas sample is exhaled breath.

34. A system for the detection of nitric oxide in a gas sample comprising:
a converter for oxidation of nitric oxide to nitrogen dioxide;
a nitrogen dioxide sensor comprising at least one nanostructure.

35. A system for the detection of nitric oxide in a gas sample comprising:
a nitric oxide sensor comprising at least one nanostructure.

36. The system of claim 35 wherein the nitric oxide sensor comprises a recognition layer in contact with the at least one nanostructure to enhance sensitivity to nitric oxide.

37. The system of claim 36 wherein the recognition layer comprises at least one polymer, a metal or a metal compound.

38. A method for detecting nitric oxide in exhaled breath comprising:
detecting nitric oxide in the exhaled breath using a nitric oxide sensor comprising at least one nanostructure.
